(19) United States
(12) Patent Application Publication
(10) Pub. No.: US 2002/0100065 A1
(43) Pub. Date: Jul. 25, 2002

(54) Production of typed human cells, tissues and organs

(76) Inventor: Esmail D. Zanjani, Reno, NV (US)

Correspondence Address: Barbara J. Luther, Chartered, Sierra Patent Group, Ltd., P.O. Box 6149, Stateline, NV, 89449, US

(21) Appl. No.: 09/895,895

(22) Filed: Jun. 29, 2001

Related U.S. Application Data
(60) Provisional application No. 60/263,927, filed on Jan. 24, 2001.

(51) Int. Cl. A01K 67/027; C12N 5/08

(52) U.S. Cl. 800/4; 800/14; 435/366

(57) ABSTRACT

A method of obtaining a high yield of differentiated human cells and organs includes the steps of providing typed human bone marrow or cord blood stem cells, providing pre-immune non-human mammalian fetuses, implanting the cells into the fetuses, permitting the fetuses to grow for a sufficient time to produce differentiated cells in hybrid organs, and harvesting the differentiated cells from the mammals.

A method is presented to produce hybrid functioning human-animal solid organs for clinical transplantations. The method includes obtaining bone marrow mononuclear cells (BMNC) from the patient, obtaining enriched populations of HSC from the BMNC, and transplanting the enriched cells into Preimmune fetal sheep or pigs intraperitoneally to produce functioning donor (patient)-animal hybrid organs. A method is also presented in which enriched HSC isolated from pre-HLA typed normal human fetal liver/bone marrow, cord blood, or bone marrow will be transplanted into preimmune fetal sheep or pigs in order to create functioning human-animal hybrid organs that can be transplanted into compatible patients. Methods are also presented to obtain high yield of different types (e.g. hepatocytes) of donor (patient or HLA-typed normal donors) cells from the human-animal hybrid organs that can be used either for transplant into patients and/or treatment of the patient.

Also disclosed is a method of producing purified human proteins that includes providing a non-human, pre-immune mammal into which human bone marrow or cord blood cells has been implanted into the mammal at the pre-immune state, obtaining blood from the non-human mammal, and isolating the human proteins from the mammalian blood.

This application claims the priority of U.S. Provisional Application 60/263,927, filed Jan. 24, 2001.

The U.S. Government may have certain rights in this invention as provided for by the terms of HL-49-042 awarded by National Heart, Lung & Blood Institute, National Institutes of Health.

### TECHNICAL FIELD

This invention is in the general field of human transplantation and specifically relates to transplantation of human cells and organs grown in a mammalian host with the intent of growing useful cells, organs and proteins for secondary transplantation to humans.

### BACKGROUND OF THE INVENTION

The hematopoietic system has long been known to harbor multipotent stem cells that self-renew and give rise to mature cells of all blood lineages. This knowledge forms the conceptual basis for bone marrow (BM) transplantation, since the stem cells present within the donor hematopoietic graft can repopulate the recipient's defective or ablated hematopoietic system. Several authors have used hematopoietic stem cells to address the question of stem cell plasticity in adult tissues, but the majority of the studies have relied on animal cells, leaving the question of whether human stem cell populations possess the same potential unanswered. In one study (Ferrari, et al. Muscle regeneration by bone marrow-derived myogenic progenitors. Science 279:1528-1530, 1998) unfractionated bone marrow cells were injected into skeletal muscle that had been chemically induced to undergo regeneration and demonstrated that the injected marrow cells were able to participate in the muscle regeneration process, giving rise to fully differentiated muscle fibers. Furthermore, the engrafted donor bone marrow participated in muscle repair if muscle degeneration was experimentally induced after BM transplantation. These studies demonstrated that BM-derived cells transdifferentiated into cells of another tissue, albeit under somewhat non-physiologic conditions. Using BM transplantation in the mdx mouse model of Duchene's muscular dystrophy, Bittner et al., (Dystrophin expression in heterozygous mdx/+ mice indicates imprinting of X chromosome inactivation by parent-of-origin-, tissue-, strain- and position-dependent factors. Anat. Embryo. 195:1 75-182, 1997) demonstrated that stem cells from mouse marrow could differentiate into skeletal muscle fibers; and even cardiac muscle cells were regenerated by the recruitment of circulating marrow-derived stem cells. Another study (Gussoni et al., Dystrophin expression in the mdx mouse restored by stem cell transplantation. Nature 401:390-394, 1999) not only confirmed the mdx mouse results, but also showed that stem cells isolated from adult skeletal muscle by Hoecsht 33342 dye exclusion—the so-called muscle side population (SP)—gave rise to dystrophin positive myofibers and muscle satellite cells, and reconstituted the hematopoietic compartment of lethally irradiated mdx recipients. In another study by Jackson, Mi and Goodell, (Hematopoietic potential of stem cells isolated from murine skeletal muscle. Proc. Natl. Acad. Sci. U.S.A. 96:14482-14486, 1999) muscle SP cells and whole BM cells were co-transplanted into irradiated murine recipients in a competitive repopulation assay with muscle SP cells. Results of this study showed that the muscle SP cells had a remarkable capacity for hematopoietic differentiation, and that they could maintain their hematopoietic potential when transferred to secondary recipients.

In addition to the above cell types, Eglitis and Mezey (Hematopoietic cells differentiate into both microglia and macroglia in the brains of adult mice. Proc. Natl. Acad. Sci. U.S.A. 94:4080-4085, 1997) demonstrated that bone marrow cells differentiated into both microglia and astroglia within the brains of murine marrow transplant recipients. And whereas the finding that microglia could derive from hematopoietic stem cells (HSC) was somewhat expected, the derivation of astroglia was not, since astroglia were generally considered to be derived from the neuroectoderm, and thus developmentally distinct from the marrow. Another example that cells from adult rodent BM can produce tissues of other embryologic derivation was reported by Petersen et al. (Bone marrow as a potential source of hepatic oval cells. Science 284:1168-1170, 1999). This study showed that stem cells from rat BM gave rise to hepatocytes in lethally irradiated rats, rescuing them by BM transplantation. Successfully engrafted recipients subsequently were treated with 2-acetylaminofluorene, a suppressor of hepatic proliferation, and carbon tetrachloride, an inducer of hepatic injury. Using three independent approaches, the investigators proved that in this system, rat donor BM cells differentiated into both ductular cells and hepatocytes.

All of the above-discussed studies relate to recipients, which were stressed and/or had no immune system (e.g., irradiated). Furthermore, all of the above-discussed studies are limited to non-human donors and recipients.

There is a need for human transplants that is far from being met. Several problems with human transplants include lack of donors, expensive typing of numerous donors, dual surgical procedures, and prolonged anti-rejection drug therapy with its attendant side effects.

Several companies are forging ahead in the xenotransplantation area but have serious problems to overcome, including organ immunogenicity and rejection. A controlled, certifiably safe supply of animal organs is required to permit the procedure to advance beyond the experimental stage.

Liver disease in particular affects many people from infancy to old age. The causes are highly varied and can include inborn errors of metabolism, poisoning, and primary and metastatic cancer. In some cases an individual may need only temporary treatment, and the patient's liver can regenerate. For example, pig livers have been used temporarily to help support the patient. In other cases, a human liver transplant is necessary.

What is lacking is a source of typed human cells and organs suitable for transplantation.

### SUMMARY OF INVENTION

It is an object of the present disclosure to provide a method of creating functioning patient-specific human-animal hybrid organs in sheep or pigs. Using the method of the present invention, it is possible to create a desired organ which has a significant number of cells derived from donor (patient) HSC. The partially humanized functioning organs can be transplanted into patients using available organ and/or cell transplant techniques.

Another object is to create organs in sheep and pigs that are at least in part composed of cells derived from HSC obtained from normal human cord blood or bone marrow of known human leukocyte antigens (HLA) types. Using the general method of the present invention, pre-HLA typed hybrid human-animal organs are made available for transplantation into patients using established medical procedures.

Further, functioning hybrid human-animal organs are produced with the disclosed invention using two approaches. In one approach, enriched HSC from patient marrow are transplanted into pre-immune sheep and/or pig fetuses to create the patient-specific human-animal hybrid organ. In the second approach, enriched HSC from pre-HLA typed normal human cord blood and bone marrow are used for this purpose.

An additional object of the present disclosure is a method of obtaining high yields of different patient-specific or pre-HLA typed human cells from transplanted sheep and pigs to be used for reintroduction into a patient or compatible human recipient.

An additional object is providing a way for producing human proteins in chimeric sheep and pigs. This method includes the creation of human-animal chimeras by the transplantation of human bone marrow or cord blood HSC into pre-immune sheep or pig fetuses, obtaining blood from the non-human mammals, and isolating the human proteins using available protein separation procedures. Preferably, the step of isolating human proteins is preceded by clotting the blood to produce serum.

Another object of the present disclosure is providing organs, tissue and cells to be used extracorporeally for uses such as detoxifying blood in a separate apparatus.

Disclosed herein is a method of obtaining a high yield of differentiated human cells that has the steps of providing typed human bone marrow or cord blood cells, providing pre-immune non-human mammalian fetuses, implanting the cells into the fetuses, permitting the fetuses to mature for a sufficient time to produce differentiated typed cells, and harvesting the differentiated typed cells from the mammals. Preferably, the mammalian fetus is a sheep or a pig. Preferably, the harvested typed cells are typed mature cells such as hepatocytes, lung cells, neurons, skin, heart muscle cells, skeletal muscle, and kidney cells.

Another embodiment includes a method of providing organs of known HLA type suitable for transplantation into humans, which has the steps of providing a non-human mammalian fetus in a pre-immune state; implanting human stem cells into the pre-immune fetus; permitting the fetuses to mature to produce a human-animal hybrid organ, and harvesting the organs from the mammals. Optimally, the typed human stem cells are obtained from cord blood or bone marrow and then typed. The harvested organ can be a liver, skeletal muscle, lung, spinal cord or kidney.

In yet another embodiment, a method of providing patient-specific organs suitable for transplantation into the patient is disclosed. The method includes the steps of providing a plurality of non-human mammalian fetuses in a pre-immune state; obtaining bone marrow from the patient; selecting from the bone marrow a plurality of stem cells; implanting the stem cells into a plurality of pre-immune fetuses; permitting the fetuses to mature for a sufficient time to produce a human-animal hybrid organ, and harvesting the organs from the mammals.

### BRIEF DESCRIPTION OF FIGURES

FIG. 1 is a schematic summarizing the procedure for transplanting human bone marrow (BM) or cord blood (CB) hematopoietic stem cells (HSC) into preimmune fetal sheep to obtain human-sheep chimeric organs.

FIG. 2 is a low-power microscopic view of liver from a normal sheep, showing no reaction with an antibody specific for human hepatocytes.

FIG. 3 is a higher-powered view of a normal sheep liver, showing the same result.

FIG. 4 is a low-powered view of a human liver, showing reaction with an antibody specific for human hepatocytes.

FIG. 5 is a higher-powered view of a human liver, showing reaction of individual hepatocytes with the antibody.

FIG. 6 is a low-powered microscopic view of liver from a sheep transplanted with 10⁵ human CD34⁺, CD38⁻ BM cells, showing human-staining hepatocytes at two months post-transplant.

FIG. 7 is a higher-powered view of the liver of a sheep transplanted as in FIG. 6, showing reaction of individual hepatocytes with the antibody.

FIG. 8 is a low-powered microscopic view of liver from a sheep transplanted with 1.1×10⁶ CD34⁻ Lin⁻ human cord blood cells, showing even greater human hepatocyte activity at 22 months post-transplant.

FIG. 9 is a higher-powered microscopic view of liver from a sheep transplanted as with FIG. 8, showing reaction of individual human hepatocytes.

FIG. 10 is a table summarizing donor (human) hematopoietic cell activity in sheep fetus transplanted with different doses of human BM CD34⁺, Lin⁻ cells.

FIG. 11 is a table summarizing hematopoietic and/or hepatic activity of human marrow CD34⁺, Lin⁻ cells in sheep given different amounts of cells showing that hepatic activity requires higher numbers of cells than that needed for hematopoietic activity.

FIG. 12 is a table summarizing hematopoietic and/or hepatic activity of human marrow CD34⁺ cells in sheep.

FIG. 13 shows two photomicrographs of livers from sheep transplanted with CD34⁺, Lin⁻ cells, and the percentages of donor (human) hematopoietic cells in bone marrow (BM) and peripheral blood (PB) of the same animals. The lower panel shows greater human hepatocyte activity in an animal exhibiting greater hematopoietic activity.

FIG. 14 shows similar types of information as in FIG. 13 at 5 weeks post-transplant (upper panel) and a lower side panel at three months post-transplant, indicating increasing human hepatocyte expansion over time.

FIG. 15 represents albumin staining of human liver and chimeric sheep liver, as well as liver of control (non-transplanted) animals. The left panels are photomicrographs of control sheep liver, the lower right panel shows a human liver, and the middle panels are livers of chimeric sheep stained for human albumin.

FIG. 16 shows analyses at 3 weeks post-transplant of livers of sheep, which received 10⁵ human BM CD34⁺, Lin⁻ cells. The upper left panel shows staining for anti-human albumin, and the lower left panel shows staining for anti-human hepatocytes. The other six graphs indicate the presence of human hematopoietic cells in sheep BM. This animal exhibited 20 ng/ml of human albumin in serum.

FIG. 17 shows the left panels of liver from sheep transplanted with two different cell types and later stained for various hematopoietic antigens. While both types produced human hematopoietic activity, only minor hepatic activity was noted.

FIG. 18 is a table summarizing donor hematopoietic and/or hepatic engraftment in vivo by different types of cells from two human sources (cord blood or BM). The numbers of “+”s indicate the relative intensity of hematopoietic or hepatic activities in bone marrow (BM) or liver of the recipient animals, respectively. Results were from three animals in each group and showed that cord blood cells were more effective than BM cells in inducing hepatic activity.

FIG. 19 is a table summarizing relative hepatic activity of human HSC from cord blood and BM. Again, cord blood cells showed more hepatic activity.

FIG. 20 is a low-powered photomicrograph of liver from sheep transplanted with 10⁵ human BM CD34⁺, 38⁻, Lin⁻ cells. There was a high degree of colonization of the sheep liver by human hepatocytes.

### DETAILED DESCRIPTION OF THE INVENTION

This invention provides a unique method of producing functioning patient-specific human-animal hybrid (or chimeric) organs in mammals, particularly sheep or pigs. Using the system of the present invention, the organ has a significant number of differentiated human cells, a pre-immune fetus having been implanted with donor (patient)-HSC. The partially “humanized” functioning organs can be transplanted into patients using available organ and/or cell transplant techniques.

Functioning hybrid human-animal organs are produced with the disclosed method using different approaches. Two examples include 1) transplanting enriched HSC from patient marrow into pre-immune sheep and/or pig fetuses to create the patient-specific human-animal hybrid organ and 2) transplanting enriched HSC from pre-HLA typed normal human CB and BM to create human-animal hybrid organs with known HLA types.

The invention also provides a method for convenient production of human cells for transfusion. Typed human cells from CB or BM are introduced into the preimmune mammalian fetus to produce differentiated human cells such as blood cells and hepatocytes, which can later be harvested and used for patient treatment. This would be particularly beneficial for the less common blood types, for universal type O or for patients who have difficulties with transfusions because of multiple antibodies.

Data shown below indicate that significant numbers of human hepatocytes were present as early as three weeks post-transplant and the hepatocytes persist and continue to colonize the chimeric liver for at least 22 months. Depending on the size of the patient, organs for transplant can be harvested as early as two months for a human fetus and neonate or later for human adults.

The invention also provides an improved method for producing human proteins. Previous methods attempting to use mammals have employed milk secretion from which protein separation can be difficult. The current method permits collection of the human protein from blood, which is easier and more effective.

The invention also provides a method of producing human-animal organs and cells for extracorporeal uses such as oxygenation or detoxifying patients' blood by utilizing the produced human organ as an extracorporeal device or as part of one. The humanized organs are expected to produce a more physiologic and less immunogenic response than animal organs or synthetic systems. The examples of recently patented extracorporeal devices in which the current inventions could be used include U.S. Pat. No. 5,976,870 to Park (liver-slide culture apparatus), U.S. Pat. No. 5,981,211 to Hu (Maintaining hepatocytes in bioreactor to treat liver failure), and U.S. Pat. No. 6,017,760 to Jauregui (detoxification by porcine hepatocytes in a perfusion device).

In two other separate studies, Brazelton et al. (From marrow to brain: expression of neuronal phenotypes in adult mice. Science 290:1775-1778, 2000) and Mezey et al. (Turning blood into brain: cells bearing neuronal antigens generated in vivo from BM. Science 290:1779-1782, 2000) showed that after lethal body irradiation, intravascularly transplanted murine BM cells migrate to the brains of adult recipient mice and differentiate into large numbers of cells expressing neuronal markers, providing further evidence that mesoderm-derived BM cells can adopt neural (ectodermal) cell fates.

In a different study (Thiese et al. Derivation of hepatocytes from bone marrow cells of mice after radiation-induced myeloablation. Hepatology 31:235-240, 2000), mice were subjected to whole body irradiation followed by BM transplantation. The presence of donor BM-derived mature hepatocytes was documented in the livers of the recipients, proving that even after minimal hepatic injury, primitive BM-derived stem cells participate in hepatocyte restoration. In a more recent study Lagasse et al., demonstrated the hepatopoietic activity of mouse HSC using a highly defined population of cells (Purified hematopoietic stem cells can differentiate into hepatocytes in vivo. Nature Med. 6:1229-1234, 2000). All of the studies cited above dealt with murine cells, except for a retrospective study of livers of patients who had received BM transplantation and had human donor BM-derived hepatocytes in the liver (Thiese et al., Liver from bone marrow in humans. Hepatology 32:11-16, 2000).

We transplanted human stem cells into fetal sheep to determine if defined populations of human hematopoietic stem cells (HSC) give rise to sufficient numbers of hepatocytes in vivo, for secondary transplantation into humans. Pre-immune fetal sheep were transplanted with either 1-5×10⁵ human BM CD34⁺, Lin⁻ cells or 1.1×10⁶ human CB CD34⁺, Lin⁻ cells intraperitoneally. The recipients were evaluated for evidence of donor (human) hematopoietic and hepatocyte activities at intervals (2 weeks to 22 months) post-transplant. All recipients exhibited significant multi-lineage human hematopoietic activity (including human CD34⁺ cells) beginning at about 1 month post-transplant; only low levels of donor hematopoietic cells were detected at 2 weeks post-transplant. Monoclonal mouse anti-human hepatocyte antibody (Clone OCH1E5), which does not recognize sheep hepatocytes, was used to detect donor hepatocytes. Low numbers of donor hepatocytes were detected at 2 weeks. At birth (i.e. 3 months post-transplant) highly significant numbers of human hepatocytes were present in all liver section. Human hepatocytes were present in numerous sites (about 13 sites/section) throughout the host liver and were also seen in close association with portal spaces. In some cases within each site, donor hepatocytes represented >80% of total hepatic cells. Overall, human hepatocytes comprised about 5-10% of the total liver cellularity in each histological section. However, in the animal transplanted with CD34⁻ cells, at 22 months post-transplant, donor hepatocytes were more uniformly distributed throughout the sections and represented about 30-40% of the total cells. These results demonstrate that defined populations of human HSC can give rise to significant numbers of hepatocytes, and prove that sheep can assay and grow human pluripotent stem cells in vivo.

Two advantages have contributed to the success of the invention and to its commercial viability. First, the mammal fetuses are quite small and can be injected with a small number of human cells, which is quite convenient. Second, weightlessness, such as in the fetal state, contributes to cell plasticity and enhances differentiation to a variety of cell types, a process which is facilitated by the continuing need of the fetus for all tissue/organ types as it grows.

Such evidence of robust production of human hepatocytes and all lineages of hematopoietic cells provides critical evidence to support several technologies. Hepatocytes are known to regenerate the liver, and if grown for an appropriate time in the recipient, are expected to form a liver suitable for transplant into a human. The further demonstration of all types of hematopoietic and lymphatic cells supports the ability of the implanted cells to differentiate in the recipient and produce large quantities of other cells suitable for transplantation. In addition, recent research also indicates the ability of HSC to generate skeletal and cardiac muscle, as well as other tissues.

Organs for human transplants can be grown in sheep or other mammals. The first reason for this is that these animals have shown an ability to grow and differentiate human cells as needed (see below). These human cells can be obtained from any newborn's CB or a patient's own marrow and typed and sorted by fluorescent-activated cell sorting (FACS) and other known techniques. For a patient-specific organ, cells from a patient's own marrow or newborn CB can be transplanted into sheep or other fetuses, such as pigs, and grown to adequate size. All or selected (i.e. the best-producing) recipients yield transplant tissue for the patient. A variety of blood types of CB can also be injected into mammalian fetuses to also produce organ transplant tissue (for example, hepatic cells). These pre-typed tissue transplants can be used to either transiently or permanently replace damaged livers. Such transplants also can be used in an apparatus such as has utilized pig liver in the past to temporarily recirculate a patient's blood to detoxify the individual (i.e., remove a poison); however, the provision of a human, typed detoxifying liver should decrease side effects.

PCR data (not shown) have indicated that transplanted HSC gave rise to differentiated cells in all other tissues and organs tested, including skin, heart, skeletal muscle, kidney, lung and neurons. This indicates that other organs and tissues besides hepatocytes and hematologic cells also are formed and supplied by the methods disclosed herein.

In addition, this method of transplantation provides an improved new method to manufacture human proteins. The finding (see below) that the sheep receiving human HSC transplants produced human albumin, which was detected in the serum, supports this. Human liver cells are also known to produce a large variety of other proteins, including some blood clotting factors. Therefore, other proteins also can be obtained by this method. The technology for large-scale separation of proteins from blood is well known in the art and offers advantages over currently available transgenic goats whose proteins need to be recovered from milk. Typically the method of obtaining all but the clotting factors is preceded by clotting the blood to produce serum.

Human BM was obtained from paid normal volunteers after appropriate, institution-approved consent. The possible risks of marrow donation and allergic reactions to iodine/local anesthetics (xylocaine) or local minor bleeding at the site of posterior iliac crest aspiration were explained. Approximately 50 normal, mature donors/year were used for studies. Male or female individuals including all minorities and a variety of ages (18-50 years) and having no known diseases, allergies or medication dependence were recruited. Qualified physicians perform the procedures. CB was obtained from normal pregnancies following appropriate institution-approved consent.

Vertebrate animals, mainly sheep, were used in these studies. Sheep were preferred for the high degree of physiologic similarity between the sheep and the human fetus during development. Furthermore, the size of the developing sheep fetus facilitates the in utero manipulations during early and middle gestation. Well-trained individuals performed all surgical procedures using appropriate institution-approved protocols.

### EXAMPLE 1

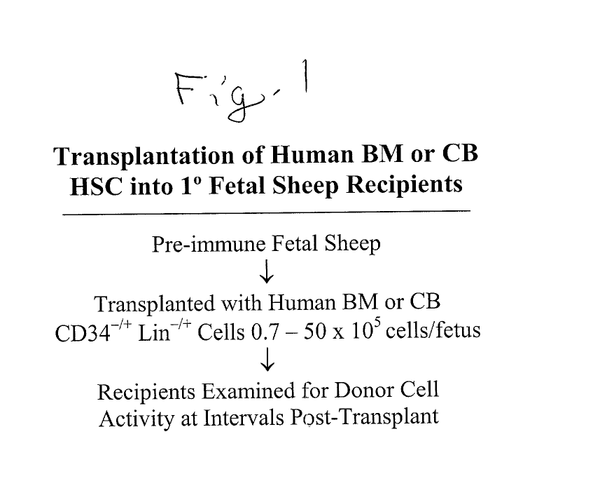

FIG. 1 summarizes the experiment. Fetal sheep were used to assess the ability of human hematopoietic stem cells (HSC) either from BM or CB to give rise to non-hematopoietic tissues including human hepatocytes. Sheep fetuses are pre-immune until about day 77 of gestation. Therefore, transplantations were carried out before this time.

Heparinized human BM was obtained from healthy donors. Low-density BM mononuclear cells were separated by Ficoll-Hypaque density gradient (1.077 g/ml) (Sigma, St. Louis, Mo.) and washed twice in Iscove's Modified Dulbecco's Media (IMDM) (Gibco Laboratories, Grand Island, N.Y.). Initially cells were enriched for CD34⁺ using the MiniMacs system (Miltenyi, Auburn, Calif.), and subsequently purified for a second antigen by using fluorescence-activated cell sorting to obtain >90% pure populations of cells specific for two antigens. Sorting of cells by other antigens (HLAA-DR⁻ or CD34⁺, Lin⁻, Thy⁺) was performed on a FACS Vantage after labeling with HLA-DR (pycoerythrin [PE]) (Becton Dickinson Immunocytometry Systems [BDIS], San Jose, Calif.) or with CD34 (Becton Dickinson), CDw 90 (5E10 fluorescein isothiocyanate [FITC]) (Pharmingen, San Diego, Calif.) and CD2, CD14, CD15, CD16, CD19 all FITC, as well as glycophorin A (AMAC, Inc., Westbrook, Me.) to exclude Lin⁺ cells as previously described. Mackay A M et al. (Chondrogenic differentiation of cultured human mesenchymal stem cells from marrow. Tissue Engineering 4:415-429, 1998); Jaiswal N. et al. (Osteogenic differentiation of purified, culture-expanded human mesenchymal stem cells in vitro. J. Cell Biochem. 64:295-312, 1997).

We transplanted pre-immune fetal sheep at 55-60 days of gestation with human BM or human CB CD34^{-/+}, Lin^{-/+} cells at 0.7-50×10⁵ cells per fetus interperitoneally using a 25 gauge needle and general techniques as described by Flake et al., Science, Vol. 233, p. 766 (1986) which permits the injection of the fetus under direct visualization in an amniotic bubble through a midline laparatomy incision. The graft was injected intraperitoneally into the fetus. The myometrium was then closed in a double layer and the pregnancy allowed to proceed. The recipients were examined for donor (human) hematopoietic and hepatocyte activities at intervals post-transplant.

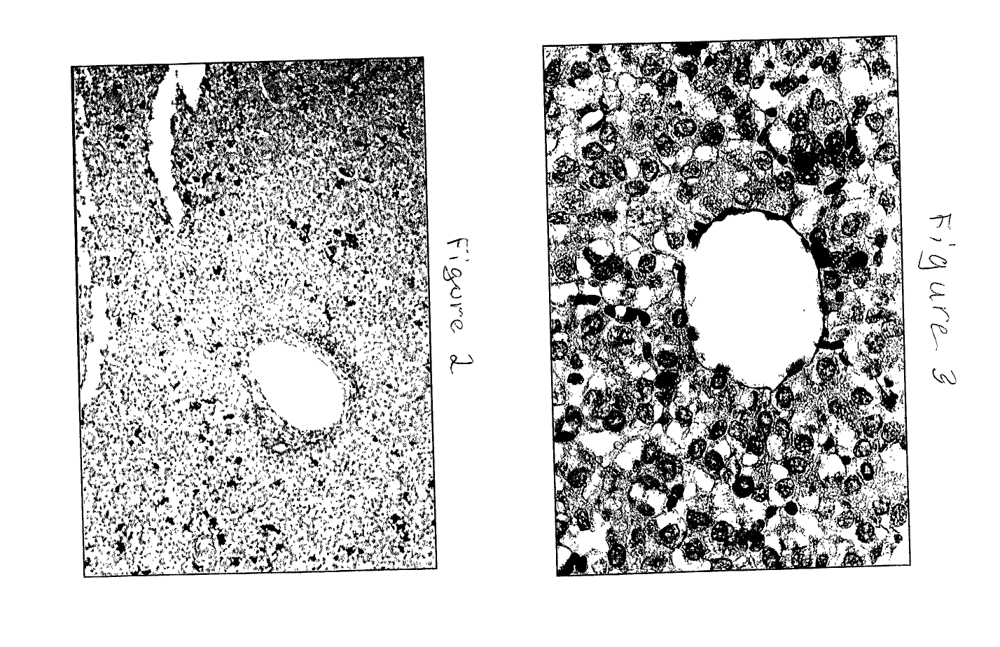

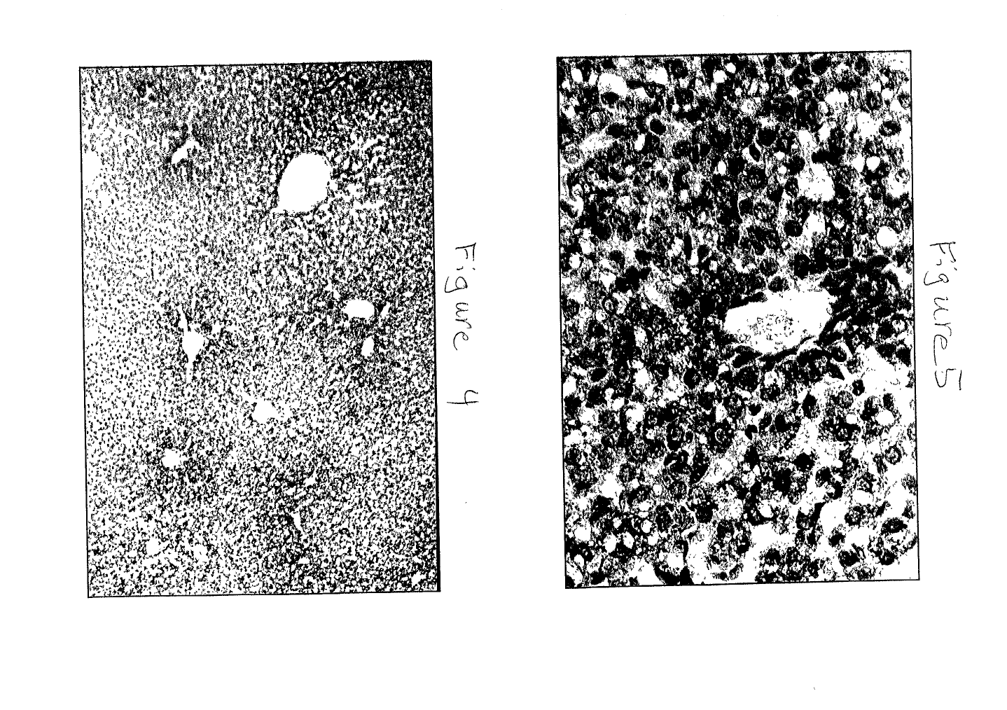

Livers from these animals were fixed in buffered formalin and embedded in paraffin. Flow cytometric analysis was performed on the peripheral blood and the BM harvested from the long bones. The presence of donor-derived hematopoiesis indicated there was successful engraftment. The livers were sectioned and stained with monoclonal antibodies specific for human hepatocytes using the commercially available anti-human hepatocyte antibody (monoclonal mouse anti-human hepatocyte antibody, clone OCH1E5, code #M7158, Lot 037, Edition 09.06.99, DAKO, Carpinteria, Calif.) and human albumin (Cloned HSA-11, Mouse ascites fluid, product #A6684, SIGMA, St. Louis, Mo.). A number of different sites of human hepatocyte activity were seen even at the low-powered view of a liver from an animal transplanted with human BM CD34⁺, Lin⁻ cells. Similar sites were also seen in slides of a liver from an animal transplanted with human BM CD34⁺, 38⁻ cells. FIGS. 2 and 3 represent negative controls from at least five untransplanted sheep livers. FIGS. 4 and 5 represent positive controls from at least three human livers, which stain with anti-human hepatocyte antibody.

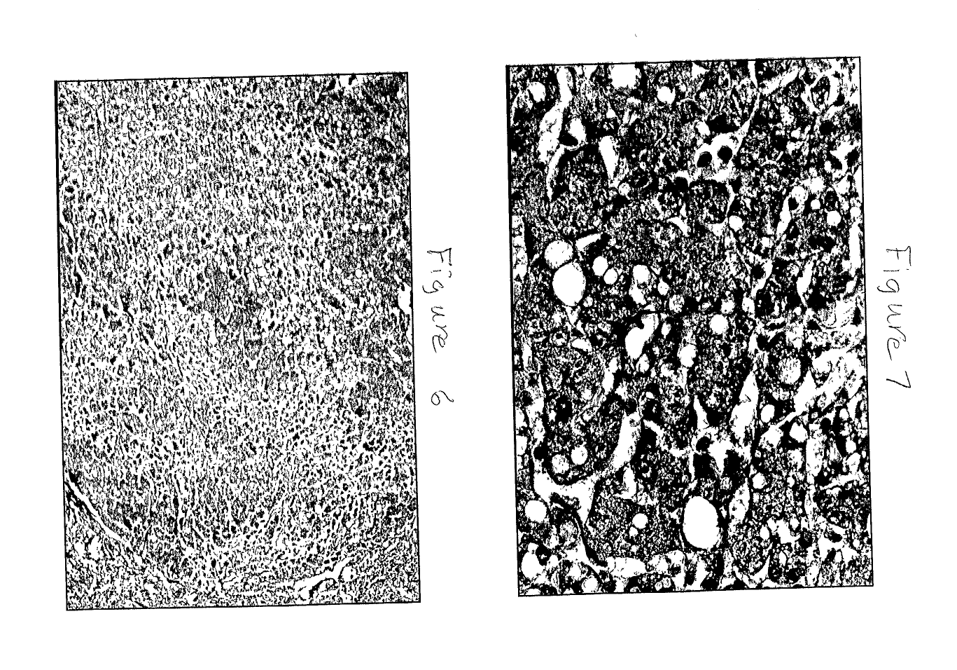

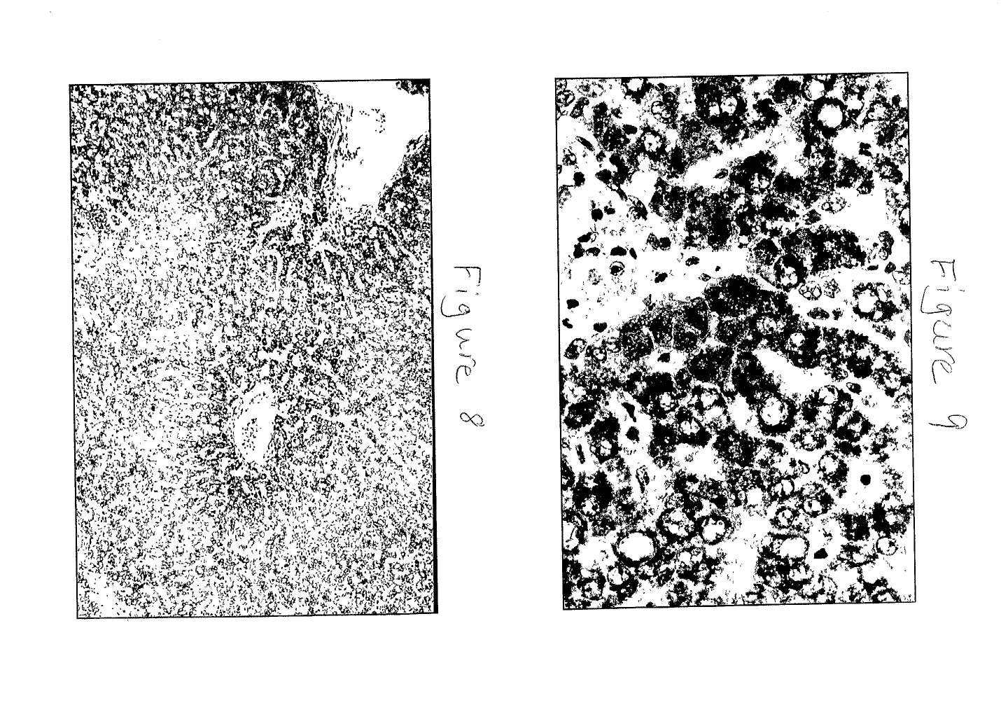

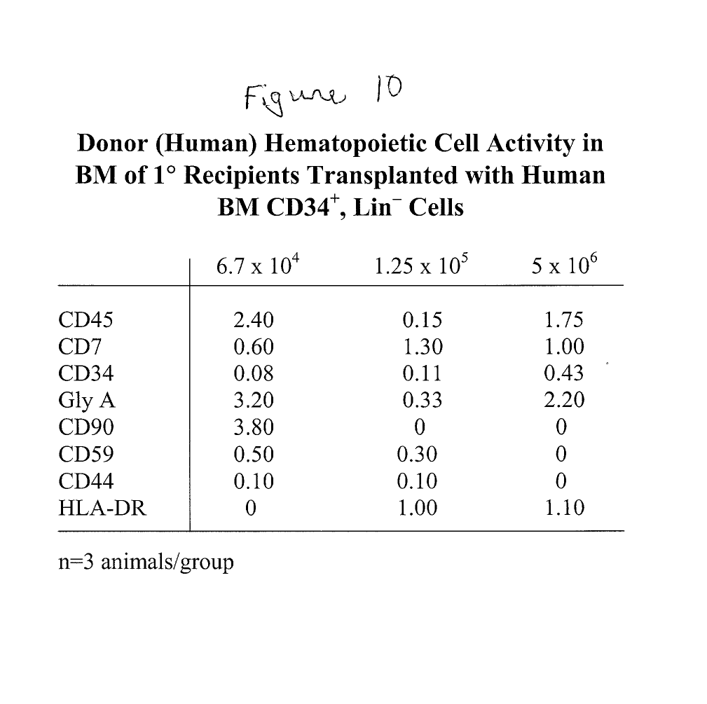

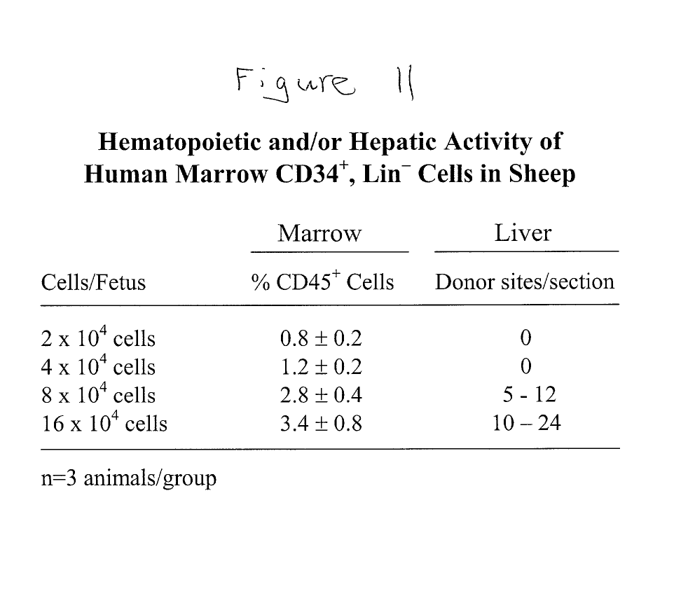

FIGS. 6 and 7 show livers from three sheep transplanted with CD34⁺, CD38⁻ BM cells. The livers showed significant hepatocyte staining in representative livers of sheep transplanted with CD34⁺, CD38⁻ BM cells. FIGS. 8 and 9 represent livers from three sheep transplanted with CD34⁺, Lin⁻ human CB cells. These livers showed even greater human hepatocyte activity. Numerous human hepatocytes were seen colonizing the sheep livers.

FIG. 10 summarizes human hematopoietic cell activity in sheep transplanted with different concentrations of human BM CD34⁺, Lin⁻ cells. These animals exhibited human hematopoietic cells of lymphoid, myeloid and erythroid origins. This is an important finding because it demonstrates that not only do human HSC engraft in this model, but they also undergo multi-lineage differentiation into all blood elements.

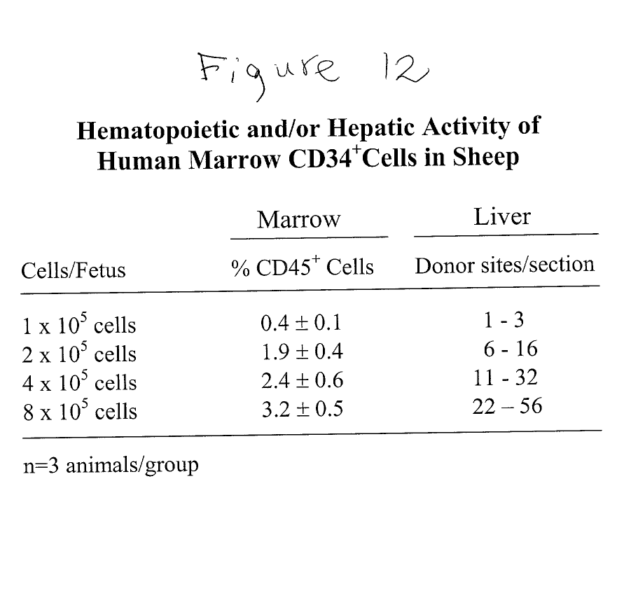

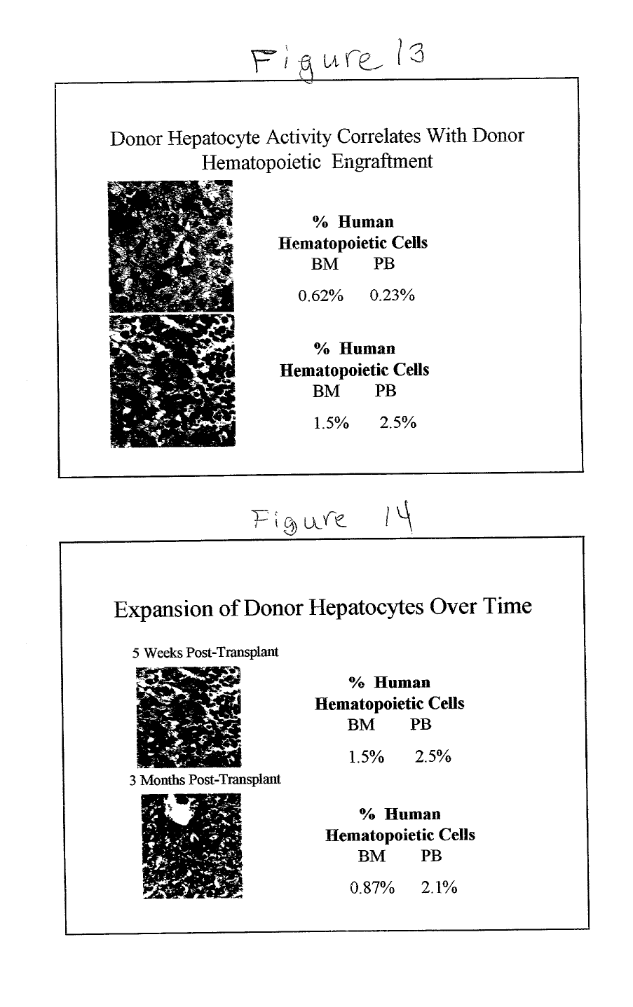

To determine the relationship between the number of cells injected and the level of human hepatocyte activity, the numbers of stem cells transplanted were correlated with the levels of human hematopoietic activity (middle column, FIG. 11) and hepatocyte colonies (right column, FIG. 11). Although human hematopoietic activity was noted with all four concentrations of human BM, CD34⁺, Lin⁻ cells, hepatocyte colonies were only detected in animals that received higher doses of cells (8×10⁴ and 16×10⁴ cells per fetus). Notably, animals transplanted at the highest HSC concentration (i.e. 16×10⁴ cells per fetus) exhibited nearly twice as many sites of human hepatocyte activity as sheep transplanted with 8×10⁴ cells per fetus. These data indicate that human hematopoiesis and human hepatopoiesis by human BM CD34⁺, Lin⁻ HSC in sheep fetuses were established by different kinetics. However, it is clear that once the threshold has been reached, as shown in FIG. 12, a direct correlation between the number of human BM CD34⁺ HSC transplanted into the pre-immune sheep fetus and the degree of human hepatocyte formation exists, supporting existence of a dose-response curve. In general, after the threshold had been reached, there was a direct correlation between the level of hematopoietic engraftment and donor hepatocyte activity (FIG. 12).

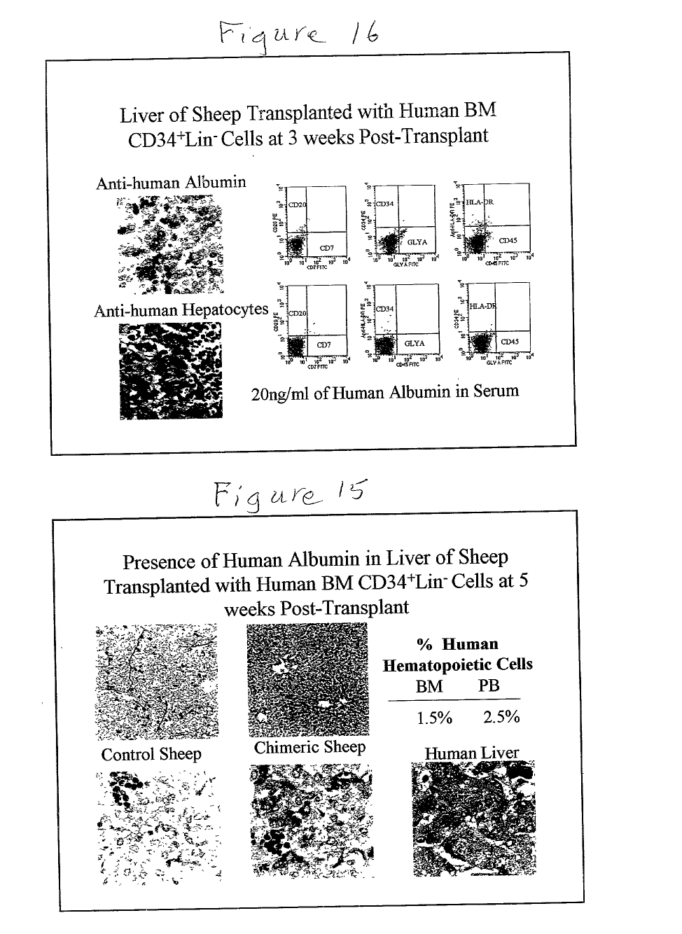

FIG. 13 reflects the hepatocyte and hematopoietic activity in a sheep transplanted with BM CD34⁺, Lin⁻ cells. The lower panel shows greater human hepatocyte activity in a sheep, which also displayed greater hematopoietic activity (lower table), which was from an animal that exhibited greater human hematopoietic activity than the animal shown in the top panel. FIG. 14 shows 5-wk and 3-mo post-transplant slides from sheep transplanted with CD34⁺, Lin⁻ cells from the same type of donor as in FIG. 13. Greater levels of human hepatocyte activity were visible in animals at 3 months post-transplant (lower panel) than at 5 weeks post-transplant, indicating increasing human hepatocyte expansion over time, even though hematopoietic expansion did not appear to increase.

### EXAMPLE 2

In this experiment, transplanted HSC produced human hepatocytes, which synthesized human albumin and were functional five weeks after transplantation. To detect human albumin we used a commercially available monoclonal anti-human serum albumin (Sigma). As can be seen in FIG. 15, liver from negative control sheep (i.e., sheep not transplanted with human HSC in utero) did not react with the antibody (left panels). By contrast, normal human liver showed a significant level of human albumin production (lower right panel). Similarly, livers from animals transplanted (chimeric sheep) with human BM CD34⁺, Lin⁻ cells exhibited positive reaction with this antibody (middle panels).

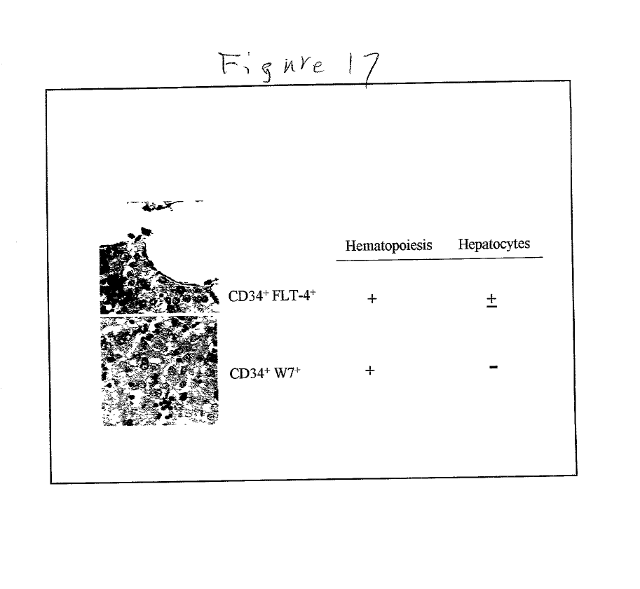

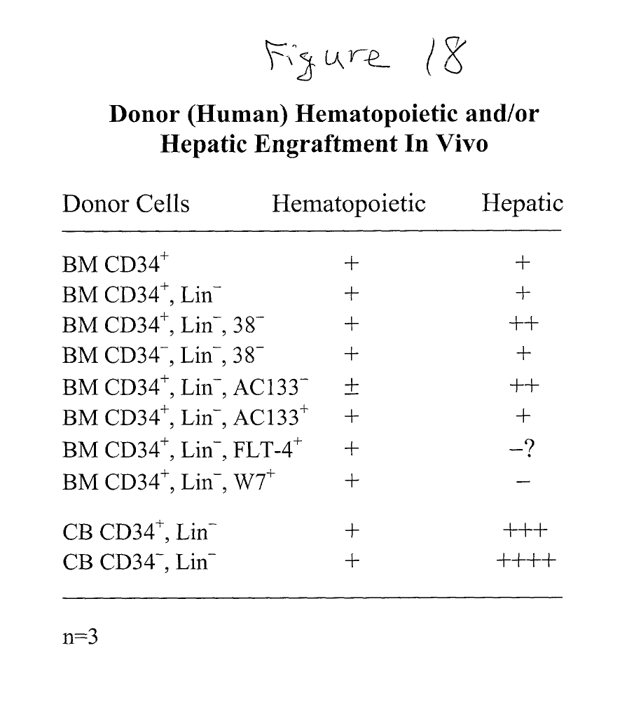

FIG. 16 shows the level of hematopoietic engraftment as well as human hepatocyte and human albumin activity (upper left panel) and human hepatocyte activity (lower left panel) in the liver of an animal transplanted with human BM CD34⁺, Lin⁻ cells at three weeks after transplant. In addition, the serum of this animal at this time contained 20 n/mL of human albumin. The presence of human albumin in the serum of this sheep was detected by ELISA (Cygnus Technologies, Wrentham, Mass.). The right six panels show the results of FACS of BM from the sheep and indicate multi-lineage hematopoietic differentiation. CD7 is indicative of thymic precursor cells, GlyA of red cells, and CD45 of leukocytes.

### EXAMPLE 3

In another experiment, different highly purified populations of human BM HSC were evaluated for their hematopoietic and hepatopoietic activity in sheep (FIG. 17). Sheep were implanted with human BM CD34⁺, FLT-1⁺ or CD34⁺, W7⁺ cells (FLT-4 and W7 antibodies obtained from Hans Buehring, University of Tubingen, Germany); and at two months post-transplant the BM and livers were evaluated for hematopoietic and hepatopoietic activity respectively. In this case there was little correlation between these two activities. Both cell types provided detectable hematopoietic activity but very little, if any hepatopoietic activity.

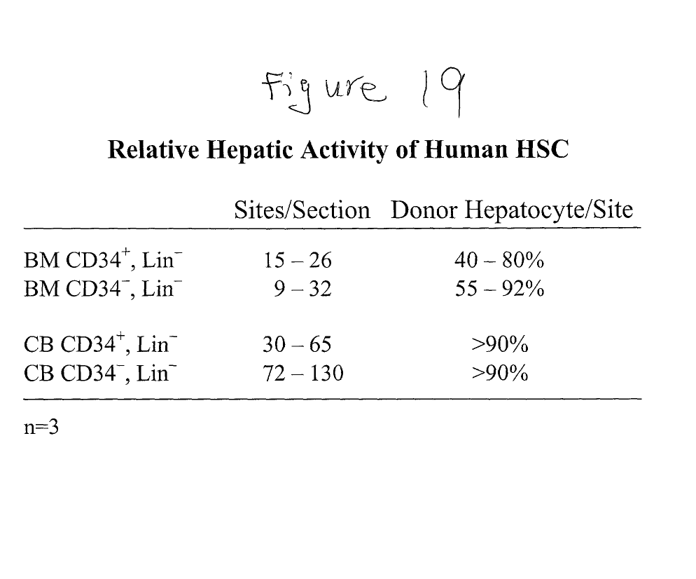

More extensive work was undertaken to more precisely identify phenotypes of human HSC with hepatopoietic potential. Ten different cell types from CB or BM were transplanted and evaluated at two months post-transplant for hematopoietic and hepatopoietic development. FIG. 18 summarizes the results of transplanting comparable numbers of human BM and CB-derived blood. The numbers of “+” in the middle and right columns indicate the relative intensity of hematopoietic and hepatic cells. The results were averaged from three animals receiving each cell type. The results presented in FIG. 18 show that human CB CD34⁺, Lin⁻ cells and human CB CD34⁻, Lin⁻ cells generated the highest level of human hepatocyte activity. However, two BM cell types also were superior in inducing hepatopoietic activity: CD34⁺, Lin⁻, 38⁻ and CD34⁺, Lin⁻, AC133. There was effective hematopoietic activity from all cell types, so any available cells can be used to induce blood formation in in utero sheep.

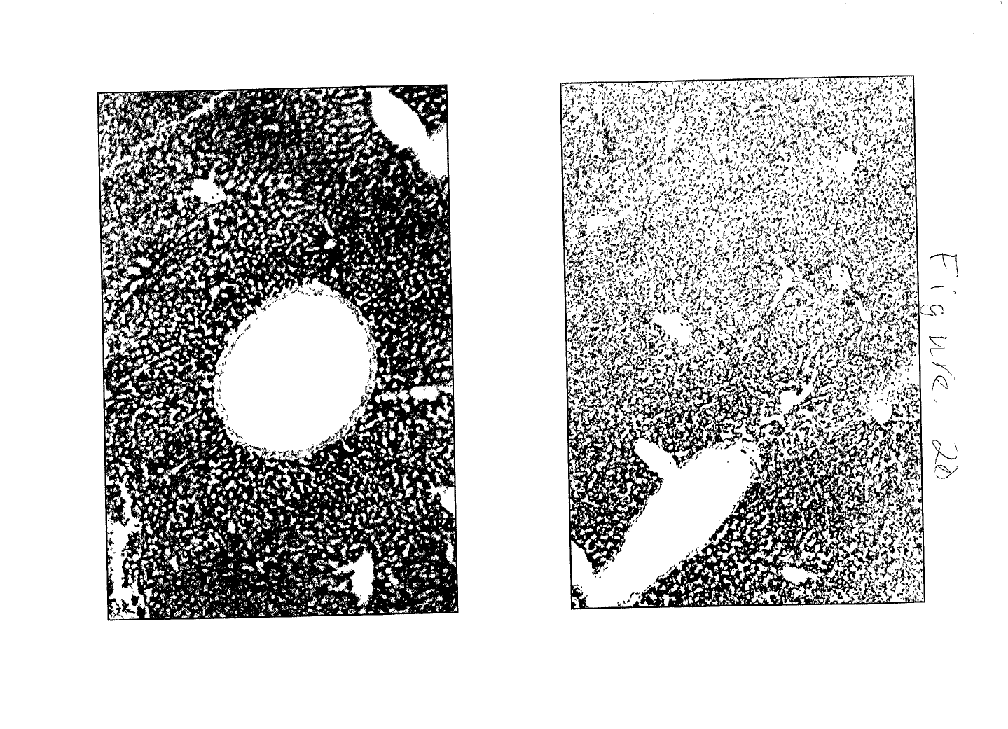

FIG. 19 shows a direct comparison of hepatopoietic activity between cells of similar phenotypes from BM and CB. These are compared by hepatic sites/section and donor hepatocytes/site therein. CB HSC of the same types exhibited significantly more human hepatocytic activity than human BM HSC. However, both cell types from BM and CB produced high levels of hepatocyte activity in the sheep livers and are suitable for injection for producing human/sheep hybrid organs.

FIG. 20 illustrates how completely the transplanted human BM cells take over the sheep liver. The sheep was implanted with 1×10⁵ CD34⁺, 38⁻, Lin⁻ cells. The sheep liver was sectioned and fixed as mentioned above, and stained with anti-human hepatocyte antibody. This representative low-powered photomicrograph shows how human HSC-derived hepatocytes have effectively colonized the sheep liver. Such a predominantly “humanized” liver would be highly desirable for transplantation, preparation of human proteins, etc.

In summary, 1) defined subsets of CD34⁺ and CD34⁻ cells from human CB and BM produce both hematopoietic and hepatic cells following transplantation into pre-immune sheep fetuses, and these hepatocytes appeared to be functional since they produced human albumin which was detectable in both the hepatocytes and circulation, 2) defined stem cell populations from human CB produced larger numbers of hepatocytes in vivo when compared to phenotypically identical cells from adult BM, 3) CD34− cells from human CB gave rise to the largest numbers of hepatocytes in vivo, 4) hematopoietic activity and hepatocyte formation were ascribed to multiple human HSC phenotypes, 5) at low human HSC doses, donor hematopoiesis occurs in the absence of hepatopoiesis, while at higher HSC doses there was a direct correlation between the number of hepatocytes formed and the dose of donor HSC.

Furthermore, these data demonstrate that defined populations of human HSC gave rise to significant numbers of functional human hepatocytes, and support the use of fetal sheep or other non-human mammals to create a ready source of human cells, tissues and organs.

The foregoing description and examples are intended only to illustrate, not limit, the disclosed invention.

1. A method of providing organs of known pre-HLA type suitable for transplantation into human, the method comprising:
a. providing a non-human mammalian fetus in a pre-immune state;
b. implanting typed human stem cells into the pre-immune fetus;
c. permitting the fetus to mature for a sufficient time to produce a human-animal hybrid organ; and
d. harvesting the human-animal hybrid organ.

2. The method of claim 1 wherein the typed human stem cells are first obtained from cord blood and then typed.

3. The method of claim 1 wherein the typed human stem cells are first obtained from bone marrow and then typed.

4. The method of claim 1 wherein the organ is a liver, lung, heart, muscle, tendon, kidney or brain.

5. A method of providing at least one patient-specific organ suitable for transplantation into a patient, the method comprising:
a. providing a plurality of non-human mammalian fetuses in a pre-immune state;
b. obtaining bone marrow from the patient;
c. selecting from the bone marrow a plurality of stem cells;
d. implanting the stem cells into a plurality of pre-immune fetuses;
e. permitting the fetuses to mature for a sufficient time to produce a human-animal hybrid organ; and
f. harvesting the organs from the mammals.

6. A method of producing at least one purified human protein, the method comprising:
a. providing a non-human, pre-immune mammalian fetus into which human stem cells from cord blood/bone marrow/peripheral blood have been implanted;
b. permitting the non-human mammal to mature;
c. obtaining blood from the non-human mammal; and
d. isolating the human proteins from the mammalian blood.

7. The method of claim 6 wherein the step of isolating human proteins is preceded by clotting the blood to produce serum.

8. The method of claim 6 wherein human albumin is isolated from the mammalian blood.

9. The method of claim 6 wherein a human clotting protein is isolated from the mammalian blood.

10. A method of obtaining a high yield of typed differentiated human cells, the method comprising:
a. providing typed human stem cells;
b. providing a plurality of pre-immune, non-human mammalian fetuses;
c. implanting the human stem cells into the fetuses;
d. permitting the fetuses to mature for a sufficient time to produce typed differentiated cells; and
e. harvesting the typed differentiated cells from the mammals.

11. The method of claim 10 wherein the human stem cells are implanted into a sheep fetus.

12. The method of claim 10 wherein the implanted stem cells are first obtained from bone marrow or cord blood.

13. The method of claim 10 wherein the typed differentiated cells are hepatocytes, neurons, muscle cells, cardiac muscle cells, skin cells, kidney cells, or lung cells.

14. Purified human proteins produced by non-human mammals implanted at the preimmune fetal stage with human stem cells.

15. Humanized organs from a non-human mammal, such organ derived by implanting human stem cells in pre-immune fetal non-human animals.
